(19) United States
(12) Patent Application Publication
(10) Pub. No.: US 2006/0293559 A1
(43) Pub. Date: Dec. 28, 2006

(54) Disposable scope cleaner and method of using same

(76) Inventors: George Daniel Grice III, Mt.Pleasant, SC (US); Joshua Cooper Miles, San Diego, CA (US)

Correspondence Address: LAW OFFICE OF PETER G. KORYTNYK. PLLC, 650A SOUTH 15TH STREET, ARLINGTON, VA, 22202, US

(21) Appl. No.: 11/165,188

(22) Filed: Jun. 24, 2005

(51) Int. Cl. A61B 1/00

(52) U.S. Cl. 600/102; 600/127; 600/157

(57) ABSTRACT

A disposable scope cleaning device and method of use are provided that allow obstructions on a distal viewing end of a medical scope encountered during an invasive medical procedure to be mechanically and fluidically removed in vivo. To achieve mechanical scrubbing and cleaning, the distal viewing end of the medical scope can be reciprocated or otherwise moved with respect to a cleaning member portion of the disposable scope cleaner. The disposable scope cleaner can include a system for flushing the distal end of the scope with an irrigation fluid. The disposable scope cleaner can be made from inexpensive, non-sterilizable materials, for example, non-autoclavable materials, so that the scope cleaner cannot be re-used and is therefore, disposable.

### FIELD

The present teachings relate to a disposable cleaning device for scopes used in invasive medical procedures. More particularly, the present teachings relate to a device and method for in vivo clearing of obstructions from the distal ends of endoscopes, laparoscopes, or any other scopes used in endoscopic surgery.

### BACKGROUND

Endoscopic surgical procedures involve a percutaneous introduction of an optical scope having an objective lens and one or more illuminating devices into a body region of a patient where a surgical procedure is to be performed. In the case of laparoscopic procedures, the viewing scope is commonly referred to as an endoscope or a laparoscope, and is commonly introduced through an access tube, such as a trocar, which provides a port into the patient. The surgical site is typically insufflated to provide a work cavity. Using a scope introduced through a trocar, a surgical team can directly view the surgical site with a camera and video monitor, allowing manipulation of a variety of specialized surgical instruments introduced percutaneously during the surgical procedure. Other conventional viewing scopes include endoscopes, arthroscopes, thoracoscopes, bronchoscopes, hysteroscopes, choledochoscopes, cystoscopes, resectoscopes, and the like.

Numerous times during surgery, blood, tissue, or other bodily material from the surgical site can adhere to a lens and/or illumination element of the endoscope thereby obstructing the transmitted field of view. Conventionally, to clear such obstructions, the scope is removed from the surgical site entirely to allow manual cleaning of the lens and/or illumination element. Such conventional practice, however, can disrupt and undesirably prolong the surgical procedure, as well as increase trauma to the patient. In addition, exposure of the objective lens to ambient room temperatures, or temperatures sufficiently below the patient's body temperature, can cause the formation of condensation on the lens when reintroducing the scope into the patient. Accordingly, the need to completely withdraw the scope from the patient, and the subsequent cleaning, reinsertion, and relocation, can be time-consuming, inconvenient, and can increase the risk of infection. Moreover, withdrawing and cleaning the scope in such a manner may not be entirely effective in enhancing the field of view of the surgical site.

To address some of the aforementioned disadvantages, it has been proposed to introduce a sleeve member between the optical scope and the trocar. Such sleeve members provide a cleaning element that can be used to mechanically clean the objective lens upon partial withdrawal of the scope from the surgical site. Other known sleeve members are made of durable materials that retain their structural and functional integrity upon being subjected to autoclaving or other harsh, post-use sterilization processes. These known devices are also expensive, complex, and complicated to manufacture.

Accordingly, a need exists for a disposable and inexpensive scope cleaning device that can improve the optical clarity of a scope without requiring the complete removal of the scope from the patient's body. A need also exists for a disposable and inexpensive scope cleaning device that can permit irrigation and warming of a scope during a surgical procedure.

### SUMMARY

An object of the invention is to solve at least the above problems and/or disadvantages and to provide at least the advantages described hereinafter.

The present teachings disclose a disposable scope cleaner comprising a hollow tube including an inner surface configured to sidably receive a viewing end of a medical scope and an outer surface configured to be slidably received within a trocar. The hollow tube can include a proximal end having a first opening and an opposing distal end having a second opening. A cleaning member can be disposed at the distal end of the hollow tube and can be configured to contact at least a portion of the viewing end of the medical scope as the medical scope is displaced in the hollow tube in a direction from the proximal end to the distal end. As the medical scope is displaced, the cleaning member can be resiliently deformed from a normal position to a distended position upon passing the medical scope through the second opening. The hollow tube and the cleaning member can be made from non-autoclavable materials.

The present teachings also disclose a disposable scope cleaner for cleaning a distal end of a medical scope in vivo. The disposable scope cleaner can include a cleaning tube including a conduit portion extending between a first extracorporeal port and an opposing second intracorporeal port. The conduit portion can be arranged to slidably receive the distal end of the medical scope. A cleaning member can be disposed at the first intracorporeal port of the cleaning tube and can be forced open as the distal end of the medical scope is directed against a surface of the cleaning member. The cleaning member can be biased closed as the distal end of the medical scope is withdrawn from contact with the cleaning member. An irrigation collar can be disposed at the second extracorporeal port of the cleaning tube and can include a channel capable of being connected to a source of irrigation fluid. The irrigation collar can be operable to direct an irrigation fluid to the cleaning member through the conduit portion. The cleaning tube, the cleaning member, and the irrigation collar can be made from non-sterilizable materials.

The present teachings also disclose a method of cleaning a distal end of a medical scope utilizing a disposable scope cleaner. The method includes proving a disposable scope cleaner including a cleaning tube having a cleaning member arranged at one end and an irrigation collar arranged at a second end. The cleaning tube, cleaning member, and irrigation collar can be made from non-autoclavable materials. The method further includes inserting the disposable scope cleaner into a trocar located at a surgical site and inserting the distal end of the medical scope through the irrigation collar and into the cleaning tube of the disposable scope cleaner. The distal end of the medical scope is slidably reciprocated with respect to the disposable scope cleaner such that a portion of the distal end of the medical scope is mechanically scrubbed by the cleaning member of the disposable scope cleaner.

Additional advantages, objects, and features of the present teachings will be set forth in part in the description which follows and, in part, will become apparent to those having ordinary skill in the art upon examination of the following or may be learned from practice of the present teachings. The objects and advantages of the present teachings may be realized and attained as particularly pointed out in the appended claims.

### BRIEF DESCRIPTION OF THE DRAWINGS

The present teachings will be described in detail with reference to the following drawings in which like reference numerals refer to like elements wherein:

FIG. 1 is a cross-sectional side view of the individual components of a scope cleaner system according to various embodiments;

FIG. 2 is a cross-sectional side view of a disposable scope cleaner after it has been inserted over a distal end of a scope according to various embodiments;

FIG. 3 is a side cross-sectional view of the arrangement of FIG. 2 after it has been inserted into a trocar at a surgical site according to various embodiments;

FIG. 4 is a side cross-sectional view of a normal operating position of the scope cleaner system showing the scope pushed through a cleaning member according to various embodiments;

FIG. 5 is an end view of the cleaning member of the disposable scope cleaner shown in FIG. 1 according to various embodiments; and

FIG. 6 is an end view of another embodiment of a cleaning member of the disposable scope cleaner.

### DESCRIPTION

Various embodiments of a disposable scope cleaner provide an apparatus and method for in vivo cleaning and warming of a lens and/or illumination device located at a distal end of a medical scope, for example, during an invasive surgical procedure. According to various embodiments, the disposable scope cleaner can be used with a wide variety of optical or viewing scopes, often referred to generally as endoscopes, and more particularly being referred to as laparoscopes, arthroscopes, thoracoscopes, bronchoscopes, hysteroscopes, choledochoscopes, cystoscopes, resectoscopes, and the like, depending on the particular type of surgical procedure being performed. The disposable scope cleaner can be used to clean the distal end of scopes during procedures where the scope is introduced into a body or intracorporeal region, for example, a patient's abdomen, to perform a surgical procedure, such as a cholecystectomy, hysterectomy, gastrostomy, appendectomy, bowel resection, herniorrhaphy, and the like. According to various embodiments, the disposable scope cleaner can scrub and remove particulate and other contamination which may physically coat at least part of the distal end of the scope during the surgical procedure. The disposable scope cleaner can also perform lens defogging by directing warm fluid onto the distal end of the scope. The condensation can form, for example, as a result of introducing a scope at room temperature into a warm, moist environment, such as, an intracorporeal surgical site.

Laparoscopic and other invasive surgical procedures are typically performed through relatively small percutaneous incisions made in the patient's abdomen or elsewhere, depending on the particular procedure being performed. Such incisions are frequently created using self-introducing, percutaneous trocars that are commercially available from any of a number of suppliers. Such trocars or guide tubes provide an access cannula having an access lumen that permits introduction of the scope and/or other appropriately-sized surgical instruments into a surgical site. The disposable scope cleaner according to various embodiments is compatible with known trocars and other percutaneous access devices, such as sheaths, dilator tubes, for example, radially expandable dilators, and the like, which can be introduced in a known manner to form and thereafter increase the diameter of percutaneous penetrations through the abdomen and elsewhere. For laparoscopic procedures in which the surgical site is insufflated with carbon dioxide, for example, the trocars can include a valve or other sealing element at their proximal ends to prevent the release of the pressurized insufflation gases. Other procedures, such as, arthroscopic and thoracoscopic procedures may not require valves or similar sealing mechanisms.

The disposable scope cleaner according to various embodiments can be used with any device or apparatus having an elongate, tubular structure defining an axial lumen which in turn provides access into a patient's body to a desired surgical site for a scope. The disposable scope cleaner according to various embodiments can also be used with scopes that do not require the use of an access device. That is, the disposable scope cleaner can be used with scopes that can be introduced directly into a natural or existing bodily orifice such as the mouth, nose, ear, anus, vagina, urethra, and the like.

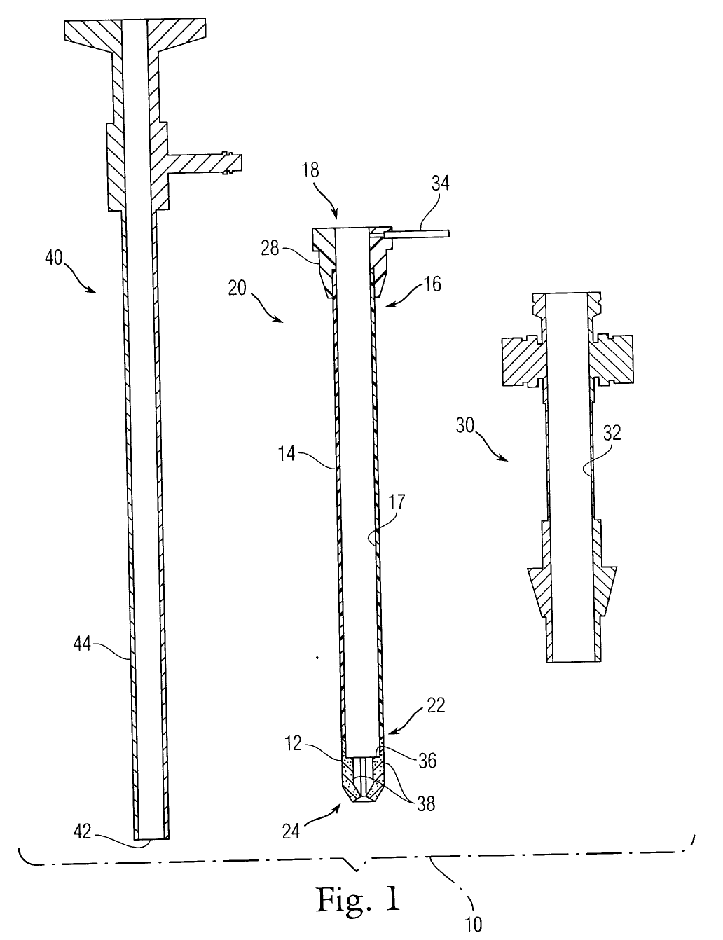

FIGS. 1-4 show a scope cleaner system 10 according to various embodiments. Referring to FIG. 1, the scope cleaner system 10 can include a disposable scope cleaner 20, a percutaneous access device 30, and a medical scope 40. According to various embodiments, the disposable scope cleaner 20 can include a thin-walled cleaning tube 14 having a first portion or proximal end 16 and an opposing second portion or distal end 22. The first portion or proximal end 16 of the cleaning tube 14 can define a first opening or extracorporeal port 18, and the opposing second portion or distal end 22 can define a second opening or intracorporeal port 24. Inner sidewall surface of the cleaning tube 14 can define a conduit portion 17 that can extend substantially from the extracorporeal port 18 to the intracorporeal port 24 and can be sized to slidably receive a distal end portion 44 of the scope 40. The distal end portion 44 of the scope 40 can include a viewing end 42 that can include one or more lenses and/or illumination devices. An outer surface portion of the cleaning tube 14 can be sized to be removably insertable and at least partially disposed within an access lumen 32 of the percutaneous access device 30.

According to various embodiments, a resilient cleaning member 12 can be mounted or disposed in the vicinity of the intracorporeal port 24 of the cleaning tube 14. Moreover, in the vicinity of the extracorporeal port 18 of the cleaning tube 14, an irrigation collar 28 can be mounted or disposed. The irrigation collar 28 can be arranged to direct a cleaning solution to the cleaning member 12, as will be discussed below.

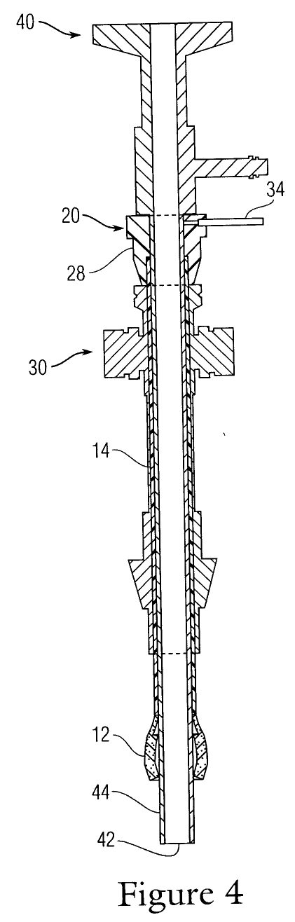

As will also be discussed below with reference to FIG. 4, as a distal viewing end 42 of the medical scope 40 is displaced in the cleaning tube 14 of the disposable scope cleaner 20 in a direction from the proximal end 16 to the distal end 22, the distal viewing end 42 can be forced against an interior surface of the cleaning member 12. As the distal viewing end 42 is forced against the cleaning member 12, the cleaning member 12 is resiliently deformed from a normal position to a distended position, thereby achieve a scrubbing, cleaning and/or defogging action on the distal viewing end 42 of the scope 40 by way of the cleaning member 12.

Referring back to FIG. 1, the cleaning tube 14, the cleaning member 12, and the irrigation collar 28 can be made from relatively inexpensive, disposable materials, allowing the cleaning device 20 to be readily disposed after use. The materials used for the disposable scope cleaner 20 can be incapable of being autoclaved and/or sterilized such that the cleaning device 20 cannot be re-used.

According to various embodiments, the cleaning member 12 can be a flexible tip that can have various shapes and/or designs. The cleaning member 12 can be a one-piece unitary member and can be friction-fit or adhered, for example, to the cleaning tube 14. The cleaning member 12 can include one or more scrubbing cleaning surfaces 36 that can be arranged on an interior surface of the cleaning member 12. Moreover, the cleaning surface 36 can include a series of ridges 38 that can promote the scrubbing of unwanted media from the distal viewing end 42 of the scope 40, as shown in FIG. 1.

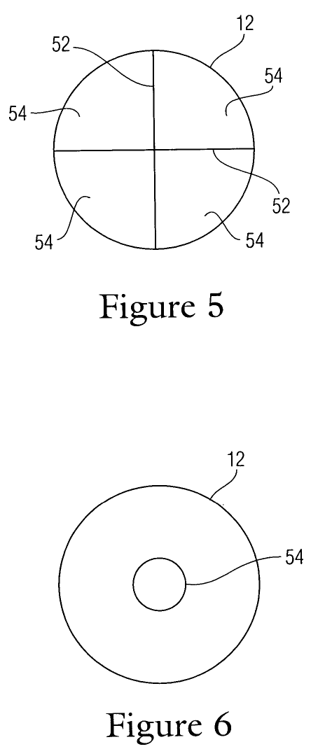

Referring to FIG. 5, which illustrates an end view of the cleaning member 12, one or more openings or slits 52 can be provided in the cleaning member 12. Accordingly, an end portion of the cleaning member 12 can be expanded and contracted as the distal end 44 of the scope 40 is passed therethrough during a surgical procedure, see FIG. 4. As the one or more slits 52 are separated or opened by the scope 40, the portions 54 of the cleaning member 12 between the slits 52, referred to as wipers, can swipe over the distal viewing end 42 and/or other portions of the scope 40, such as, for example, an illuminating element and/or lens. The interior cleaning surfaces 36 of the wipers 54 thereby scrub and displace debris, condensation, contaminants, and other media that can become lodged on the distal end 44 of the scope 40. The cleaning member 12 can include two or more wipers 54 depending on the number of slits 52 provided in the cleaning member 12. For example, as shown in FIG. 5, the cleaning member 12 can include four wipers 54. According to various embodiments, a single slit can be provided around the circumference of the cleaning member 12 to form a single wiper that can be hinged to the remaining portion of the cleaning member 12.

Alternatively, as shown in FIG. 6, the pliable cleaning member 12 can be provided with an aperture 54 that can expand as the distal end 44 of the scope 40 is forced against the cleaning member 12. At a certain point, the distal end 44 of the scope 40 can be forced through the aperture 54 thereby scrubbing and displacing debris, condensation, contaminants, and other media that can become lodged on the entire distal end 44 of the scope 40.

According to various embodiments, the disposable scope cleaner 20 can be provided with an irrigation collar 28 for directing a cleaning or flushing solution to the cleaning member 12 to help flush away and remove the debris, condensation, contaminants, and other media from the distal end 44 of the scope 40. The irrigation collar 28 can include one or more attachment tubes 34 for connection with various tubing structures and various types of fluid sources. The attachment tubes 34 can be located at or in the location the irrigation collar 28 and can extend any distance and at any angle from the irrigation collar 28. The irrigation collar 28 and the one or more attachment tubes 34 can be a one-piece unitary member. The irrigation collar 28 can be friction-fit or adhered, for example, to the cleaning tube 14.

A fluid source can provide an irrigation fluid to the disposable cleaning device 20 that can convey the irrigation fluid to the cleaning member 12 through an annular space that can be formed between the disposable scope cleaner 20 and the scope 40. The irrigation fluid can be any fluid, such as, for example, saline solution, or any other fluid suitable for flushing debris away from and/or controlling a temperature of the scope 40 in whole or in part. According to various embodiments, the irrigation fluid can be delivered to the disposable cleaning device 20 at a temperature that can be higher or lower than room temperature.

According to various embodiments, at least one of the attachment tubes 34 can be attached to a vacuum source that can be used to evacuate liquid and unwanted media from the distal end 44 of the scope 40 during use.

According to various embodiments, the cleaning member 12 can be made from a resilient material, such as, for example, an open cell or surgical foam. Moreover, the cleaning member 12 can be impregnated or saturated with a suitable cleaning solution, such as a mild, physiologically compatible or inert detergent. The material of the cleaning member 12 can be capable of receiving and/or applying at least one of the irrigation fluid and the cleaning solution to the distal end 44, and in particular, the distal viewing end 42 of the scope 40, to clean and/or defog the one or more lenses and/or illumination devices.

The cleaning member 12 can offer sufficient resistance to the passage of the scope 40 so that the user can determine when contact between the distal viewing end 42 and the cleaning member 12 has been established. The user can thus engage the distal viewing end 42 of the scope 40 against the cleaning member 12 and can rotate or otherwise purposefully manipulate the distal viewing end 42 against the cleaning member 12, for instance, to produce a scrubbing or other effect, for cleaning the distal viewing end 42 to a desired degree of clarity.

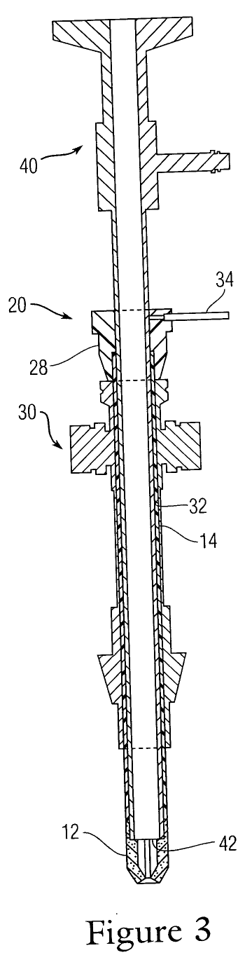

According to various embodiments, the cleaning tube 14 can be an elongate rigid structure. Moreover, the cleaning tube 14 can be sized such that it is capable of being inserted into the access lumen 32 of a conventional access device 30 while simultaneously receiving a conventional scope 40 within the conduit portion 17. Accordingly, as shown in FIGS. 3 and 4, the cleaning tube 14 can be sized as to be interposed between the access device 30 and the scope 40, for example, in a nested arrangement of components of the scope cleaner system 10.

The cleaning tube 14 can be arranged to occupy as little area of the access lumen 32 of the access device 30 as possible so as to only minimally increase the effective diameter of the scope 40 as it is introduced through the access lumen 32 of the access device 30. According to various embodiments, the cleaning tube 14 can be a thin-walled sleeve, having a wall thickness of about 0.5 mm or less. The wall thickness of the cleaning tube 14 can depend on the internal dimensions of the access device 30 into which the cleaning tube 14 is to be disposed, as well as the dimensions of the scope 40 that is inserted into the cleaning tube 14. For example, conventional trocars can have inner diameters of about 12.2 mm. Accordingly, the cleaning tube 14 can have an outer diameter of just below about 12.2 mm and an inner diameter of about 12 mm so as to accommodate a number of commercially available scopes 40.

According to various embodiments, the overall dimensions of the disposable scope cleaner 20 will be longer than that of the access device 30 into which the disposable scope cleaner 20 is to be arranged.

According to various embodiments, the cleaning member 12, the cleaning tube 14, and/or the injection collar 28 can be made from any rigid, semi-rigid, collapsible, non-reusable, disposable, physiologically suitable material, formed by extrusion techniques, injection molding and other processes. Examples of suitable materials can include polytetrafluoroethylene (e.g., TEFLON®), polymer, plastic, resin, and the like, and any combination thereof. The materials can be non-sterilizable, for example, non-autoclavable. For example, the components of the disposable scope cleaner 20 can be made of materials that lose functional or structural integrity, for instance, when exposed to a sufficient amount of heat, for example, materials having a melting point of about 132° C. or less, for example, of from about 120° C. to about 140° C. The materials can be nonmetallic and/or not containing any metal or metallic alloy.

According to various embodiments, the cleaning member 12, the cleaning tube 14, and/or the injection collar 28 can be made from the same or different materials. According to various embodiments, the disposable scope cleaner 20 can be made of multiple pieces or of a one-piece unitary construction. For example, the disposable cleaning device 20 can be a one-piece unitary construction that changes rigidity from a proximal end to a distal end, wherein the proximal end of the disposable cleaning device 20 is rigid and the distal end is relatively soft such that it can clean the distal end of the scope without damaging the scope.

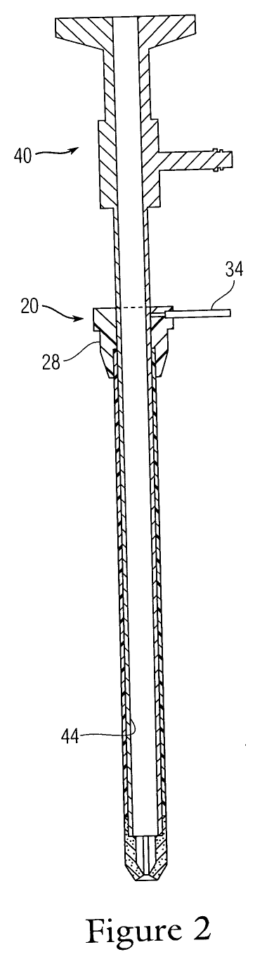

FIGS. 2-4 show the use of the disposable scope cleaner 20 in more detail. Referring to FIG. 2, the disposable scope cleaner 20 is shown after it has been slid over and inserted onto a distal end 44 of the scope 40. At this point, the combination of the scope 40 and disposable scope cleaner 20 can be inserted and used at a surgical site. FIG. 3 shows a surgical site where a trocar 30, having an access lumen 32, can be used to permit the introduction of the scope 40 and the disposable scope cleaner 20 into the surgical site. As shown in FIG. 3, the irrigation collar 28 of the disposable scope cleaner 20 can engage the trocar 30 and can form a pressure seal therebetween. FIG. 4 shows a normal operating position of the scope 40, whereby the distal end 44 of the scope 40 is pushed through the cleaning member 12, thereby resiliently deforming and opening the cleaning member 12 by outwardly expanding the slits 52 formed therein. As shown in FIG. 4, the irrigation collar 28 of the disposable scope cleaner 20 can engage the scope 40 and can form a pressure seal therebetween. In the position illustrated in FIG. 4, the distal viewing end 42 of the scope 40 can be used to view the surgical site.

The use of the disposable scope cleaner 20 detailed above discloses one contemplated order of use. It is contemplated that the disposable scope cleaner 20 according to various embodiments can be used in various other manners. For example, the disposable scope cleaner 20 could be first inserted into a trocar 30 and then the distal end 44 of the scope 40 could be subsequently inserted into the disposable scope cleaner 20.

During the surgical procedure, whenever the distal viewing end 42 becomes contaminated with debris, fog, or other obscuring media, the distal viewing end 42 can be cleaned by withdrawing the scope 40 in a direction away from the surgical site, so that it can re-enter into the interior of the cleaning member 12, as shown in FIG. 3. In a possible cleaning position as shown in FIG. 3, the cleaning member 12 can close and surround the distal viewing end 42. The distal viewing end 42 can be scrubbed by reciprocating the scope 40 between the positions shown in FIGS. 3 and 4. During such reciprocating cleaning movement, the inner surface of the cleaning member 12 can wipe across and scrub the distal viewing end 42 of the scope 40. Moreover, the scope 40 can be rotated or manipulated in infinite ways against the cleaning member 12, for example, when in the position shown in FIG. 3, to produce a scrubbing or other effect to clean the distal viewing end 42 or any portion of the distal end 40 to a desired degree of clarity. In such ways, for example, the inner surface or any portion of the cleaning member 12 can act as a cleaning surface.

According to various embodiments, before, during, and/or after performing the reciprocating and/or rotary cleaning movement, an irrigation fluid can be directed to the cleaning member 12 to help flush away and remove debris, condensation, contaminants, and other media. Irrigation fluid can be provided to the irrigation collar 28 by way of a fluid source that is in fluid communication with the attachment tube 34. Irrigation fluid can be conveyed to the cleaning member 12 through an annular space between the disposable scope cleaner 20 and the scope 40. Similarly, debris, condensation, contaminants, and other media can be removed from the surgical site by connecting a vacuum source to an attachment tube.

Such a cleaning operation can be-repeated as often as desired during a surgical procedure to keep the distal viewing end 42 of the scope 40 free of debris. Upon completion of the surgical procedure, the disposable scope cleaner 20 can be withdrawn from the patient and discarded.

The disposable scope cleaner 20 according to various embodiments advantageously provides alternative devices and methods for cleaning surgical optical scopes in vivo, i.e., without the need to remove the viewing scope from the patient. Such devices and methods need not require modification of the scope, and can require minimum or no modification of other instruments used in performing the surgical procedure, for example, trocars used for introducing the scope. Such devices and methods are effective in removing contaminating debris and fog from the distal viewing end of the scope, are convenient to use, and are low-cost, due, in part, to the simple low-cost construction and disposability of the scope cleaner.

Those skilled in the art can appreciate from the foregoing description that the present teachings can be implemented in a variety of forms. Therefore, while these teachings have been described in connection with particular embodiments and examples thereof, the true scope of the present teachings should not be so limited. Various changes and modifications may be made without departing from the scope of the teachings herein.

What is claimed is:

1. A disposable scope cleaner comprising:
a hollow tube including an inner surface configured to slidably receive a viewing end of a medical scope and an outer surface configured to be slidably received within a trocar, the hollow tube including a proximal end having a first opening and an opposing distal end having a second opening; and
a cleaning member disposed at the distal end of the hollow tube and configured to contact at least a portion of the viewing end of the medical scope as the medical scope is displaced in the hollow tube in a direction from the proximal end to the distal end such that the cleaning member is resiliently deformed from a normal position to a distended position upon passing the medical scope through the second opening;
wherein the hollow tube and the cleaning member are made from non-autoclavable materials.

2. The disposable scope cleaner of claim 1, wherein the scope cleaner is a unitary, one-piece device.

3. The disposable scope cleaner of claim 2, wherein the scope cleaner changes rigidity from the proximal end to the distal end whereby the proximal end is rigid and the distal end including the cleaning member is soft such that the distal end is operable to clean the medical scope without damaging the scope.

4. The disposable scope cleaner of claim 3, wherein the scope cleaner is made from a polymer.

5. The disposable scope cleaner of claim 1, further comprising an irrigation collar disposed at the proximal end and configured to define a portion of an irrigation channel that extends through the irrigation collar to the cleaning member.

6. The disposable scope cleaner of claim 5, wherein the irrigation collar is configured to define a portion of an evacuation channel extending from the cleaning member to the irrigation collar for connection to a vacuum device for removing fluid and debris from the cleaning member.

7. The disposable scope cleaner of claim 5, wherein the irrigation collar includes a seal such that a pressurized seal is formed by an engagement between the irrigation collar and the trocar.

8. The disposable scope cleaner of claim 1, wherein the cleaning member includes a series of ridges that are operable to scrub the viewing end of the medical scope as the cleaning member is resiliently deformed from the normal position to the distended position.

9. A disposable scope cleaner for cleaning a distal end of a medical scope in vivo, comprising:
a cleaning tube including a conduit portion extending between a first extracorporeal port and an opposing second intracorporeal port, the conduit portion being arranged to slidably receive the distal end of the medical scope;
a cleaning member disposed at the first intracorporeal port of the cleaning tube and arranged to be forced open as the distal end of the medical scope is directed against a surface of the cleaning member and to be biased closed as the distal end of the medical scope is withdrawn from contact with the cleaning member; and
an irrigation collar disposed at the second extracorporeal port of the cleaning tube and including a channel capable of being connected to a source of irrigation fluid, the irrigation collar being operable to direct an irrigation fluid to the cleaning member through the conduit portion;
wherein the cleaning tube, the cleaning member, and the irrigation collar are made from non-sterilizable materials.

10. The disposable scope cleaner of claim 9, wherein the cleaning member is a unitary, one-piece member.

11. The disposable scope cleaner of claim 10, wherein the surface of the cleaning member comprises sponge or pliable material that is operable to scrub the medical scope as the medical scope is contacted with the cleaning member.

12. The disposable scope cleaner of claim 9, wherein the irrigation collar includes a second channel capable of being connected to a vacuum source, the irrigation collar forming a portion of an evacuation channel extending from the cleaning member to the irrigation collar for for removing fluid and debris from the cleaning member.

13. The disposable scope cleaner of claim 9, wherein the cleaning tube, the cleaning member, and the irrigation collar are made from materials having a melting point of less than about 132° C.

14. The disposable scope cleaner of claim 9, wherein the scope cleaner is a unitary, one-piece device.

15. The disposable scope cleaner of claim 14, wherein the scope cleaner changes rigidity from the irrigation collar to the cleaning member whereby the irrigation collar is rigid and the cleaning member is soft such that the cleaning member is operable to clean the medical scope without damaging the scope.

16. The disposable scope cleaner of claim 15, wherein the scope cleaner is made from a polymer.

17. A method of cleaning a distal end of a medical scope utilizing a disposable scope cleaner comprising:
proving a disposable scope cleaner including a cleaning tube having a cleaning member arranged at one end and an irrigation collar arranged at a second end, the cleaning tube, cleaning member, and irrigation collar being made from non-autoclavable materials;
inserting the disposable scope cleaner into a trocar located at a surgical site and inserting the distal end of the medical scope through the irrigation collar and into the cleaning tube of the disposable scope cleaner; and
slidably reciprocating the distal end of the medical scope with respect to the disposable scope cleaner such that a portion of the distal end of the medical scope is mechanically scrubbed by the cleaning member of the disposable scope cleaner.

18. The method of claim 17, further comprising directing an irrigation fluid through the irrigation collar, between the cleaning tube and medical scope, and to the cleaning member.

19. The method of claim 18, further comprising removing the distal end of the medical scope and the disposable scope cleaner from the trocar and throwing away the disposable scope cleaner.
